(19) United States
(12) Patent Application Publication
(10) Pub. No.: US 2004/0126830 A1
(43) Pub. Date: Jul. 1, 2004

(54) Test strip and method for determining LDL cholesterol concentration from whole blood

(76) Inventors: Bruce Shull, Indianapolis, IN (US); Hyeon-Sook Lee Zeng, Indianapolis, IN (US); Sunil Anaokar, Indianapolis, IL (US); Gena Lynn Antonopoulos, Indianapolis, IN (US)

Correspondence Address: MAGINOT, ADDISON & BOWMAN, BANK ONE CENTER/TOWER, 1111 MONUMENT CIRCLE, SUITE 3000, INDIANAPOLIS, IN, 46204, US

(21) Appl. No.: 10/663,555

(22) Filed: Sep. 16, 2003

Related U.S. Application Data
(60) Provisional application No. 60/411,209, filed on Sep. 16, 2002.

(51) Int. Cl. C12Q 1/60; G01N 33/558

(52) U.S. Cl. 435/11; 436/514

(57) ABSTRACT

A dry phase test strip (20) and method are provided for determining the concentration of LDL in whole blood or plasma. The inventive test strip (20) includes one stack (92) or panel that measures concentration of total cholesterol and another stack (94) or panel that measures concentration of the sum total of HDL, VLDL and chylomicrons (“non-LDLs”). The difference between the values just noted is equal to the concentration of LDL cholesterol. Dry phase test strips (20) of the present invention function at room temperature and all test results are produced from pseudo-endpoint reflectance measurements such that the test method need not be timed. Also disclosed is the capability for an improved lipid panel that provides concentration in a blood sample of HDL, total cholesterol and LDL cholesterol without relying upon the Friedewald equation.

### REFERENCE TO RELATED APPLICATION

The present application claims priority to co-pending provisional application Serial No. 60/411,209, bearing the same title, which was filed on Sep. 16, 2002. The disclosure of this application is incorporated herein by reference.

### FIELD OF THE INVENTION

The present invention relates generally to testing of body fluids for concentration of analytes and more particularly to test strips for determining concentration of analytes in whole blood.

### BACKGROUND

The level of cholesterol in blood is a significant indicator of risk of coronary heart disease. “Total cholesterol” includes low density lipoproteins (LDL), very low density lipoproteins (VLDL) and high density lipoproteins (HDL). It is well-established from epidemiological and clinical studies that there is a positive correlation between levels of LDL cholesterol (“bad” cholesterol) and coronary heart disease and a negative correlation between levels of HDL cholesterol (“good” cholesterol) and coronary heart disease. Standing alone, the level of total cholesterol in blood, which is a measure of the sum total of HDL, LDL, VLDL and chylomicrons, is not generally regarded as an adequate indicator of the risk of coronary heart disease because the overall level of total cholesterol does not reveal the relative proportions of HDL, LDL and VLDL. To better assess the risk of heart disease, it is desirable to determine the amount of LDL cholesterol in addition to total cholesterol.

The most common approach to determining LDL cholesterol in the clinical laboratory is the Friedewald calculation, which estimates LDL cholesterol from measurements of total cholesterol, HDL cholesterol and triglycerides. Although convenient, the Friedewald calculation suffers from several well-established drawbacks. Nauck et al., Methods for Measurement of LDL-Cholesterol: A Critical Assessment of Direct Measurement by Homogeneous Assays versus Calculation, Clin. Chem. 48:2 (2002) (citation omitted). For example, since the Friedewald calculation involves measurements other than LDL cholesterol, it is subject to potential compounded inaccuracies from the determinations of the other lipids in the equation. Further, its usefulness is limited when assaying blood samples with triglycerides levels above 400 mg/dl.

Ultracentrifugation is a known technique to separate LDL cholesterol, but it is tedious, time consuming, and the highly labile lipoproteins can be substantially altered by the high salt concentrations and centrifugal forces. “Furthermore, a plethora of different types of equipment and tubes are used, making conditions difficult to reproduce from one laboratory to another and consistent separations highly dependent on the skills and care of the technician.” Id. at 238.

Other techniques for measuring LDL cholesterol include electrophoresis, which requires a fresh agarose gel specimen, is only semi-automatic, and depends at least in part on the technique of the technician performing the test. Other so-called homogeneous methods have recently become available.

One homogeneous method for determining LDL is disclosed in U.S. Pat. No. 5,888,827 (Kayahara, Sugiuchi, et al.; assigned to Kyowa Medex Co., Japan). The '827 patent discloses a two-stage liquid phase reaction to quantify LDL concentration in a fluid sample. In the first step, the sample containing LDL cholesterol is placed in a first reagent which includes trimethyl β-cyclodextrin as a sugar compound, polyoxyethylene monolaurate as a protein solubilizing agent, EMSE (N-ethyl-N-(3-methylphenyl)-N′-succinylethylenediamene) and Tris buffer. The sample is then heated to 37° C., and after 5 minutes the absorbance is read. A second reagent including cholesterol esterase, cholesterol oxidase, peroxidase, 4-aminoantipyrine and Tris buffer is then added and after another 5 minutes the absorbance is again measured at the same wavelength. LDL cholesterol is then calculated by separately subjecting a standard solution of cholesterol to the same procedure and comparing the respective absorbance values. This method suffers from the drawback of requiring conducting the reaction at a temperature of 37° C. Further, this method requires individual reagents to be added in two distinct steps at two different times

Another two-stage homogeneous assay is disclosed in U.S. Pat. No. 6,194,164 (Matsui et al.; assigned to Denke Seiken, Ltd. Japan). In the first stage, HDL, VLDL and chylomicrons in the test sample are “erased,” and in the second step, the cholesterol remaining in the test sample (viz., LDL) is quantified. In the first step, cholesterol esterase and cholesterol oxidase act on the test sample in the presence of a surfactant which acts on lipoproteins other than LDL (“non-LDLs”). The hydrogen peroxide thereby generated is decomposed to water and oxygen by catalase. Alternatively, a phenol-based or an aniline-based hydrogen donor compound is reacted with the hydrogen peroxide to convert it to a colorless quinone. Preferred surfactants which act on the non-LDLs include polyoxyethylene lauryl ether, polyoxyethylene cetyl ether, polyoxyethylene oleyl ether, polyoxyethylene higher alcohol ether, polyoxyethylene octyl phenyl ether, polyoxyethylene nonylphenyl ether, and the like. In the second reaction disclosed in the '164 patent, cholesterol remaining in the test sample, which should theoretically contain only LDL, is quantified. The second step may be carried out by adding a surfactant which acts on at least LDL and quantifying the hydrogen peroxide by the action of the cholesterol esterase and the cholesterol oxidase added in the first step.

Like the '827 patent, one disadvantage of the method taught by the '164 patent is that it requires conducting the reaction at a temperature of 37° C., and it has been found that the test is not accurate at lower temperatures. Another disadvantage of the '164 patent similar to the '827 patent is that the '164 patent requires individual reagents to be added in two distinct steps at two different times. A more general disadvantage of these liquid phase LDL tests is that they are not easily adaptable to point of care (“POC”), much less over the counter (“OTC”) applications.

What is needed, then, is a convenient, easy to use, diagnostic test for determining LDL cholesterol which overcomes the drawbacks noted above.

### SUMMARY OF THE INVENTION

The present invention provides a dry phase test strip and method for determining the concentration of LDL in whole blood or plasma. The test strip directly measures concentration of total cholesterol and directly measures concentration of non-LDLs, the difference therebetween being equal to the concentration of LDL cholesterol. Dry phase test strips of the present invention function at room temperature and all test results are produced from pseudo endpoint reflectance measurements, such that the test method does not require timing.

In one form thereof, the present invention provides a method of determining concentration of non-LDL cholesterol in a whole blood sample using a dry phase test strip. The whole blood sample is contacted with a blood separation layer of the test strip and the blood cells are separated from the sample, thereby producing plasma. The plasma so produced is then contacted with a test layer and the non-LDL fraction reacts substantially faster than the LDL fraction to produce color substantially in proportion to the concentration of LDL cholesterol in the sample. The color produced is measured and corresponds to the concentration of non-LDL cholesterol. The test layer employs a surfactant which acts on lipoproteins other than LDL, “non-LDLs,” such that the non-LDLs react to produce color and the result is read photometrically before any LDLs have substantially reacted. In this manner, the reflectance or colorimetric response is a function of non-LDL concentration, substantially unaffected by LDL concentration.

By reading the concentration of non-LDLs and also reading concentration of total cholesterol, the concentration of LDL cholesterol can be easily calculated as the difference therebetween.

In another form thereof, the present invention provides a test strip for determining the concentration of LDL cholesterol in a sample of whole blood or plasma. The test strip includes a test strip matrix having at least two stacks. A first one of the stacks has reagents incorporated therein to produce a calorimetric response in proportion to the amount of total cholesterol in the sample. A second one of the stacks has reagents incorporated therein to produce a color response in proportion to the amount of non-LDL cholesterol in the sample. The value of non-LDL cholesterol obtained from the second stack can be subtracted from the value of total cholesterol obtained from the first stack to yield the value of LDL cholesterol in the sample.

One advantage of the present invention is that it provides a dry phase test strip that produces reliable measurements of LDL cholesterol without relying on the Friedewald equation. As noted above, the Friedewald equation has serious drawbacks.

Another advantage of the present invention is that the inventive test strips can be used over a range of room temperatures, quite unlike the known homogeneous liquid LDL assays, which require heating to 37° C. This temperature independence of the present invention is a significant advantage because test strips required to be heated to a specified temperature would be largely unmarketable in the over the counter (“OTC”) and point of care (“POC”) markets and, of course, inconvenient. The temperature independence of the present invention was a surprising result, in that the prior art liquid LDL assays and the testing of the same strongly suggests that temperature must be tightly controlled to produce reliable and accurate results in the liquid phase.

Yet another advantage of the present invention is that, even though a two stage reaction occurs in the non-LDL stack or panel of the strip, the first stage being production of color in proportion to non-LDLs and the second being the production of color in proportion to LDL cholesterol, the reactions need not be timed. Instead, a “pseudo endpoint” is produced at the completion of the first stage, whereupon color production in proportion to non-LDL cholesterol concentration is read by an optoelectronic instrument. Not having to time the reaction is a significant advantage that allows the test strips to have much wider applicability, ease of use, and reliability than would otherwise be possible. The time independence of the present invention was quite surprising, in view of the prior art liquid LDL assays all teaching timed reactions.

Still another advantage of the present invention is that the non-LDL test is completed and the result read in less than 1 1/2 minutes. Thus, with test strips in accordance with the present invention that read multiple analytes, e.g., HDL, total cholesterol and non-LDL, the three results are all obtained at about the same time. Waiting for the non-LDL result is unnecessary. The present single measurement approach is in contrast to the prior art LDL assays noted above that teach two-stage reactions and a result measured after both stages.

Still another advantage of the present invention is that it provides a dry phase “lipid panel” which measures total cholesterol, HDL cholesterol and LDL cholesterol results without reliance upon the Friedewald equation, and without the need to measure triglycerides, which was hitherto not possible in a dry phase test strip.

To summarize, the present invention provides a dry-phase test strip for determining LDL concentration in whole blood or plasma that is inarguably quicker, more convenient, and is essentially time and temperature independent. This breakthrough technology makes possible for the first time the potential for point-of-care and patient self-testing of LDL cholesterol without relying upon the Friedewald equation.

### BRIEF DESCRIPTION OF DRAWINGS

The above-mentioned and other advantages of the present invention, and the manner of obtaining them, will become more apparent and the invention itself will be better understood by reference to the following description of the embodiments of the invention taken in conjunction with the accompanying drawings, wherein:

FIG. 1 is a perspective view looking down on an assembled and closed test strip in accordance with the present invention;

FIG. 2 is an exploded perspective view of a test strip holder in accordance with the present invention, the view being taken from the bottom of the test strip holder;

FIG. 3 is perspective view of a test strip holder in accordance with the present invention, the test strip holder having its top and bottom portions unfolded and the underside thereof being shown;

FIG. 4 is an exploded perspective view of a test strip holder in accordance with the present invention illustrating the layers and stacks of the test matrix and their relationship with the top and bottom portions of the test strip holder;

FIG. 5 is a side sectional view of an exemplary test matrix in accordance with one embodiment of the present invention;

FIG. 6 is a graph illustrating color production versus time for the two stage reaction that occurs in the non-LDL stack of panel of test strips in accordance with the present invention;

FIG. 7 is a perspective view illustrating the vertical flow scheme utilized by the stacks and blood separation layer of the present invention;

FIGS. 8, 9 and 10 are cross-sectional views of the layers of test strips used in certain of the examples given hereinbelow; and

FIGS. 11 and 12 are cross-sectional views of the layers of test strips in accordance with alternate embodiments of the present invention.

Corresponding reference characters indicate corresponding parts throughout the several views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

### Definitions

“HDL” refers to high density lipoprotein.

“LDL” refers to low density lipoprotein.

“VLDL” refers to very low density lipoprotein.

“NonHDL” refers to LDL, VLDL and chylomicrons, i.e., lipoproteins other than HDL that will react with a conventional cholesterol reaction membrane.

“Non-LDL” refers to HDL, VLDL and chylomicrons, i.e., lipoproteins other than LDL that will react with a conventional cholesterol reaction membrane.

“Plasma” refers to the non-cellular portion of blood from which cellular components such as red blood cells are excluded.

“Serum” technically differs from plasma, in that it does not include fibrinogen. However, for purposes of this application “serum” and “plasma” are sometimes used interchangeably.

“Room Temperature” means from about 17° C. to about 30° C.

### Test Device

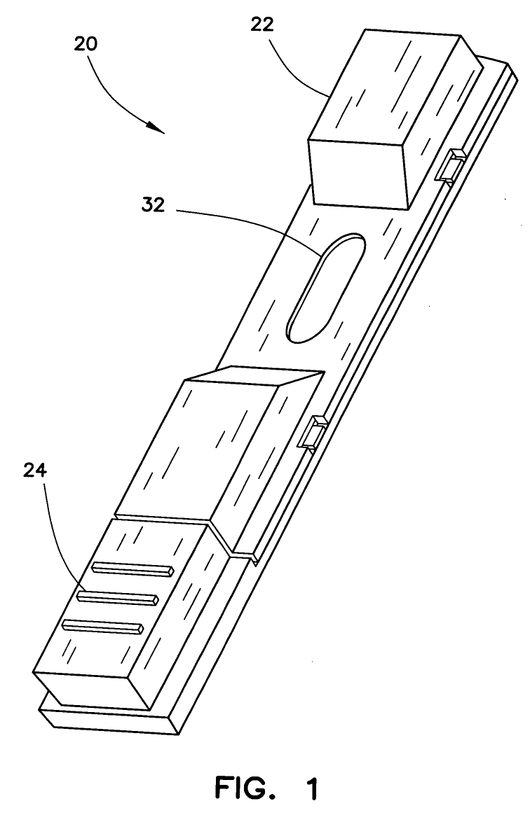

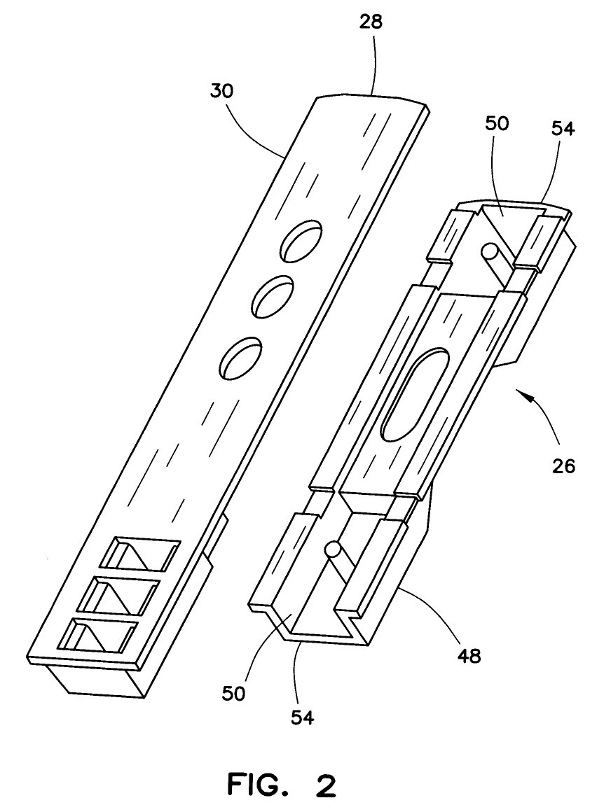

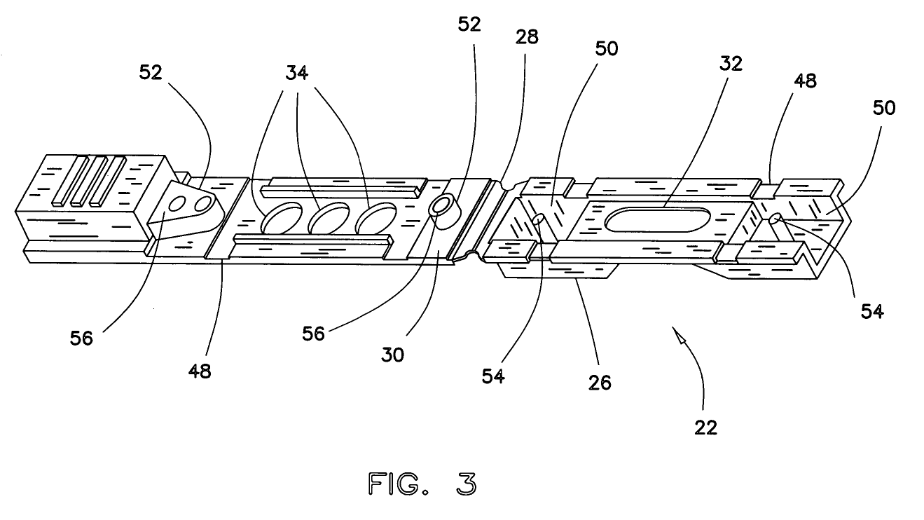

Referring now to FIG. 1, test strip 20 includes test strip holder 22 which is preferably formed by injection molding. Test strip holder 22 includes handle 24 and top portion 26 (FIGS. 2 and 3) which is preferably hingedly attached by hinge portion 28 to bottom portion 30, shown exploded away in FIG. 2. With reference to FIG. 3, top portion 26 is foldable about hinge portion 28 over bottom portion 30 as shown. Top portion 26 includes an opening 32 while bottom portion 30 includes three spaced openings 34. Opening 32, while shown as round, can be formed as an elongated oval shape to facilitate disbursement of blood. When top portion 26 is folded over bottom portion 30, opening 32 is aligned centrally over openings 34. In its folded position, opening 32 in holder 22 defines an area for depositing a body fluid sample while openings 34 define areas in which optoelectronic measurements of chemistry test reactions are conducted. Optionally, openings 34 can be configured with transparent windows, although such is not necessary.

The particular test strip described herein is suitable for use with a modified optoelectronic instruments sold under the trademarks BioScanner and Cardio Chek, available from Polymer Technology Systems, Inc., Indianapolis, Ind.

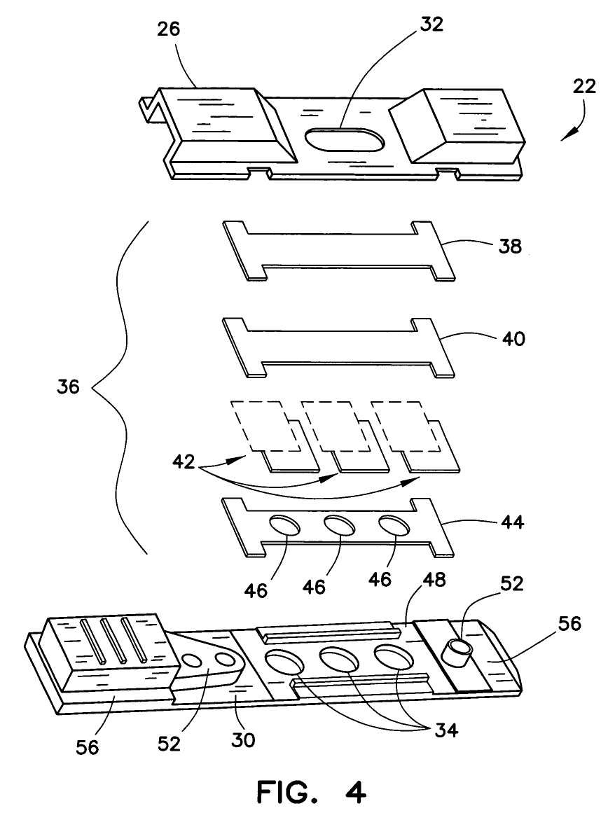

Referring now to FIG. 4, top and bottom portions 26 and 30 of strip holder 22 sandwich a test matrix 36 therebetween. Test matrix 36 is made up of a top disbursement layer 38, a blood separation layer 40, stacks 42, and adhesive layer 44 having openings 46 that align with openings 34 and the bottoms of respective stacks 42 when the layers are assembled. Stacks 42 are further made up of one or more vertically aligned layers, the function and specifics of which are described in further detail hereinbelow. The second layer of the “stacks” 42 in FIG. 4 is shown in phantom to indicate that this second layer is not used in all embodiments disclosed in this application. When assembled and closed, the layers of stacks 42 and layers 38, 40 and 44 are all pressed together. Opening 32 exposes a part of disbursement layer 38 and openings 34 and 46 expose the bottom surface of the bottom layers of stacks 42.

It has been found that only a minimally compressive force provided by strip holder 22 is necessary to sandwich the layers of test matrix 36. To this end, portions 26 and 30 have complementary I-shaped indentations or recesses 48 (FIGS. 2 and 3) in which the corresponding I-shaped matrix 36 is received. Recesses 48 allow portions 26 and 30 to be snapped together in a snap-tight engagement as shown in FIG. 1 while still exerting a minimally compressive force on matrix 36. As shown in FIGS. 2 and 3, top portion 26 includes oval shaped receptacles 50 that receive complementary shaped bosses 52 disposed on portion 30. Receptacles 50 include pegs 54 that fit via friction fit into mating cylindrical openings 56 formed in bosses 52. Stacks 42 all include the same number of layers or at least have about the same thickness, such that the bottom surfaces of stacks 42 are substantially coplanar. This coplanar structure helps maintain the proper compressive pressure on matrix 36 by holder 22.

It should be understood that at the time of this writing, it is believed that a minimally compressive force exerted upon matrix 36 is preferable. However, the amount of pressure with which matrix 36 is to be pressed together is a design variable that can be adjusted by (1) adjusting the depth of recesses 48; (2) adjusting the engagement between receptacles 50 and bosses 52; or (3) adjusting the height of pegs 54 and/or the depth of cylindrical openings 56.

Referring to FIG. 5, the individual layers and the diagnostic chemistries of matrix 36 can be appreciated. The top layer of matrix 36 is a disbursement or spreader layer capable of rapidly spreading the blood sample 58 rapidly through layer 38 as shown by the reference arrows. It has been found that layers used as conjugate pads in pregnancy test kits perform quite well as layer 38. Layer 38 is an open cell layer capable of rapidly and effectively spreading the fluid sample. One suitable material for layer 38 is available under the name “Accuflow Plus-P” from Schleicher & Schuell, Inc. Another suitable material for layer 38 is available under the name “Accuwik” from Pall Biochemicals. Both of these layers are made of polyester and provide excellent movement of blood sample 58 through layer 38.

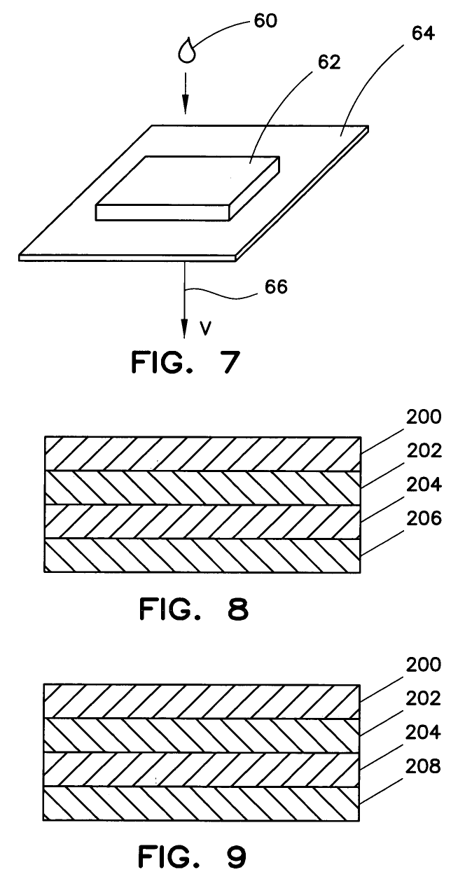

As will become clearer with reference to the discussion below, substantial lateral movement occurs only in disbursement layer 38 of matrix 36. As shown by the reference arrows in FIG. 5, however, the delivery of blood from layer 38 to layer 40 occurs substantially vertically, or normal to the plane of layer 40. In the remaining layers, the net direction of fluid flow is believed to be substantially vertical, or normal to the plane of the layers. For example, with reference to FIG. 7, fluid sample drop 60 is deposited onto layer 62 (which could be blood separation layer 40 or one of the layers from one of stacks 42). Layer 62 defines a plane 64 that is substantially parallel therewith. Transfer of fluid through layer 62 is normal or perpendicular to plane 64, or in the direction of vector V, shown at reference numeral 66. Thus, there is no substantial migration of fluid from one side of layer 62 to the other. Fluid flow is through layer 62, not across it.

Returning now to FIG. 5, layer 40 is a blood separation layer that is adjacent to and in fluid communication with layer 38. Blood separation layer 40 separates blood cells from plasma and passes the plasma therethrough, retaining the blood cells. Blood separation layer 40 is generally a glass fiber membrane. A suitable commercial membrane for layer 40 is Ahlstrom Grade 144, thickness 0.378 mm, available from Ahlstrom Filtration, Inc., Mt. Holly Springs, Pa. Other glass fiber matrices could be substituted as demonstrated in the examples that follow hereinbelow. More specifics regarding the blood separation membrane are given in co-pending U.S. provisional patent applications serial Nos. 60/344,300 and 60/342,790, commonly owned by the assignee of the present invention, the disclosures of which are hereby incorporated by reference herein in their entirety.

### Total Cholesterol Measurement Stack

With further reference to FIG. 5, the stack 92 is formed of a single layer and is spaced from stack 94 and is adjacent to and in fluid communication with layer 40. Stack 92 takes plasma from layer 40 and produces a color response proportional to the concentration of total cholesterol in sample 58. In the embodiment depicted in FIG. 5, stack 96 is also a “total cholesterol” stack, identical to stack 92. The preparation of reagents for the total cholesterol stacks (also called “panels”) is set forth in detail in the examples hereinbelow.

### Non-LDL Stack

Still referring to FIG. 5, middle stack or layer or panel 94 forms a color response that is proportional to the amount of non-LDL cholesterol in sample 58, at room temperature and without requiring the reaction to be timed, as will be explained below. Layer 94 is loaded with reagents such that non-LDL cholesterol produces a color response much faster than does LDL cholesterol. The preparation of reagents for the non-LDL stack or panels is set forth in detail in the examples hereinbelow.

The non-LDL cholesterol layer 94 differs from the cholesterol layer primarily in that layer 94 includes a surfactant which acts on non-LDLs, i.e., lipoproteins other than LDL. A commercially available and suitable surfactant is sold under the trade name Emulgen B66. Generally, however, it is believed that many other polyalkylene oxide derivatives having HLB values of between 13 and 15 would work suitably as the surfactant. Examples include condensation products with higher alcohols, condensation products with higher fatty acids, condensation products with higher fatty acid amides, condensation products with higher alkylamines, condensation products with higher alkylmercaptane and condensation products with alkyl phenols.

### Test Methodology and Theory

By taking the total cholesterol concentration derived from one or the average of stacks 92 and 96 and subtracting therefrom the amount of non-LDL cholesterol measured in stack 94, the amount of LDL cholesterol can be obtained.

More particularly, the reaction that produces color from non-LDL cholesterol is significantly faster than the reaction that produces color from LDL cholesterol, particularly at the lower room temperatures used by the present invention. Surprisingly, it has been found that an optoelectric instrument which uses a pseudo end-point algorithm, as disclosed in U.S. Pat. No. 5,597,532 effectively detects such endpoint after the non-LDLs in the plasma provided to layer 94 have reacted, but before the LDL cholesterol has significantly contributed to color production. That is, an end-point can be detected while the reaction producing color from LDL cholesterol is ongoing. The “pseudo end-point” is reached when there is only a small change in color density per time interval, e.g., five (5) seconds. For example, the algorithm can be programmed to reach a pseudo endpoint when change in reflectance drops to less than 1% over 5 seconds. This pseudo endpoint chemistry allows measuring the reflectance and thus non-LDL concentration without timing the reaction, which is a significant advantage.

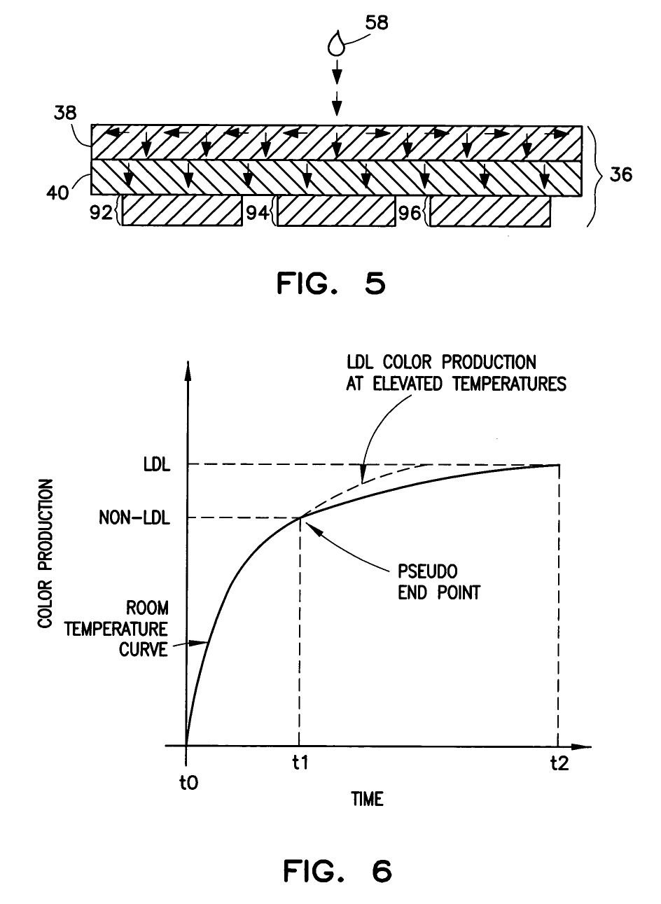

With reference to FIG. 6. At time t₀, layer 94 becomes wetted with plasma and the non-LDLs in the sample begin to produce color quickly as shown by the curve in FIG. 6. At time t₁, (pseudo endpoint shown on the curve), the nonLDLs have substantially completely reacted to form color, but the LDL cholesterols have not. Nonetheless, the reaction of the LDLs after t₁ is much slower. Consequently, the algorithm detects an endpoint at the time the slope flattens as shown at t₁.

The difference in reaction rates of non-LDLs versus LDLs produces a “pseudo endpoint” sufficient to form a cut-off point for the algorithm, which is a significant and surprising advantage. It is significant in that there is no need to establish a predetermined time which corresponds to the completion of non-LDL color production. It is surprising because the liquid phase tests, from which the dry phase tests were adapted, required strict control of the time at which the non-LDL measurement was taken, which is consistent with the homogeneous prior art assays described above that require the first phase to be timed.

Without wishing to be tied to any specific theory, it has been found that the pseudo endpoint is enhanced by conducting the test at lower temperatures, viz., room temperature, in stark contrast to the prior art teachings of heating the liquid reagents to 37° C. Lower temperatures are believed to inhibit the slow phase (LDL color production) sufficiently such that the slope of the LDL production curve shown in FIG. 6 is sufficiently flat. Yet, at the same time, the first phase of the reaction, in which the non-LDLs are expended to produce color, is nonetheless sufficiently fast and ends sufficiently abruptly such that the pseudo endpoint shown in FIG. 6 is always detected at room temperature.

On the other hand, if the test strip in accordance with the present invention were used at elevated temperatures, e.g., 37° C., as taught by prior art liquid phase tests, the second stage LDL reaction would take place more quickly, as reaction kinetics are typically enhanced by higher temperatures. A faster second phase is desirable in the liquid phase tests discussed above because it shortens the total test time, which even at 37° C. can be quite long. However, with the present invention, at elevated temperatures, it has been found that the pseudo endpoint is not as prominent and can therefore be missed by the algorithm. See dashed line corresponding to elevated temperatures in FIG. 6. Thus, the elevated temperatures taught by prior art liquid phase LDL assays teach away from the present invention. Indeed, testing of commercially available prior art homogeneous LDL measurement devices has demonstrated that their accuracy is significantly reduced if the test is conducted at temperatures even a few degrees lower than 37° C. Further, the precise times required to perform the tests in the liquid phase also teach away from the invention, whose end-point for non-LDL production can be determined by an endpoint algorithm, without having to time the reaction In view of the unacceptable results produced by the homogeneous LDL tests at temperatures lower than 37° C., it was quite surprising that the inventive dry phase test strip can be used at room temperature and over a range of temperatures.

Quite unlike the prior art liquid phase tests, which teach two separate measurements at two subsequent times, the present inventive dry phase test strips never measure LDL concentration directly. Thus, the length of time required to complete the second phase of the reaction, in which LDLs react to produce color, is not important, whether it be 2 minutes or 20 minutes. Further advantageously, since this novel test strip does not require the LDL concentration to be directly measured, only a single step of the two phase reaction occurring in test layer 94 is measured, thereby completely eliminating one of the two sequential measurements taught and indeed required by certain of the prior art homogeneous assays discussed above.

Another important discovery is that the selectivity of layer 94 for non-LDLs is dependent upon pH of the solution which impregnates layer 94 to a greater extent than in the liquid solutions used in homogeneous liquid phase tests. As detailed in the examples hereinbelow, pH of the impregnating solution of layer 94 should be pH 7. Selectivity for non-LDLs decreases as the pH becomes lower than 7.

### EXAMPLES

Specific examples embodying the technology described above are set forth below.

### Example 1

Example 1 illustrates the adaptation of the test from the liquid phase and the reliance on pH.

Spectrophotometric assay of LDL Cholesterol: pH 5 vs. pH 7 Formulations:

| | |
|---|---|
| Reagent 1a | 100 mM Citric Acid, pH 5 |
| | 0.5% Triton X-100 |
| | 0.56 mM MAOS |
| Reagent 1b | 100 mM Citric Acid, pH 5 |
| | 0.5% Emulgen B66* |
| | 0.56 mM MAOS |
| Reagent 1c | 100 mM MOPS Buffer, pH 7.0 |
| | 0.5% Emulgen B66* |
| | 0.56 mM MAOS |
| Reagent 2 | 100 mM Citric Acid, pH 5 |
| | 1.5 kU/mL Cholesterol Oxidase |
| | 4 kU/mL Peroxidase |
| | 4.8 kU/mL Cholesterol Esterase |
| | 4 mM 4-Amino antipyrine |

*Emulgen B66 is a nonionic surfactant available from Kao Corporation (1-3, Bunk 2-Chome; Sumida-Ku; Tokyo 131-8501, Japan). Emulgen B66 has previously been shown to have a selective action for the reactivity of Non-LDL Cholesterol (cited in US; U.S. Pat. No. 6,194,164). Triton X100 is used as a nonselective surfactant to give a Total Cholesterol Reaction.

In the assay, 8 μL EDTA plasma were added to 750 μL of Reagent 1 and incubated for 5 minutes at 37° C. The reaction was initiated with 250 μL Reagent 2 and scanned on a Bio-Spec1601 spectrophotometer (Shimadzu) for 200 seconds at 630 nm. A 200 mg/dL calibrator was used with Reagent 1a (pH 5.0/Triton X100) and Reagent 2 to obtain a factor for the calculation of concentration

Total cholesterol was measured with Reagent 1a (pH 5/Triton X100) and non-LDL cholesterol was measured separately using either Reagent 1b (pH 5/Emulgen B66) or 1c (pH 7.0/Emulgen B66). Measurements were made at various pre-selected times after initiating the reaction with Reagent 2. The optimum Reaction time was determined to be 75 seconds. LDL Cholesterol was calculated as the difference between Total and Emulgen B66 reactive Cholesterol.

Reference LDL Cholesterol was measured using a commercially available Kit: LDL Direct Liquid Select Cholesterol Reagent. This kit was run according to the manufacturer's directions using a Cobas Mira clinical analyzer. This reaction is performed in two steps . In the first step sample is mixed with a reagent that solubilizes only non-LDL Cholesterol. During this step, non-LDL is removed with a reaction that does not produce color. Then a second reagent is added to produce color with the remaining LDL Cholesterol.

| | | |
|---|---|---|
| Sample | Reference LDL | Measured LDL |

pH 5 Data with 75 Second Measurement Interval

| | | |
|---|---|---|
| 1 | 75 | 58 |
| 2 | 127 | 70 |
| 3 | 128 | 85 |
| 4 | 101 | 68 |
| 5 | 182 | 111 |

pH 7 Data with 75 Second Measurement Interval

| | | |
|---|---|---|
| 1 | 75 | 86 |
| 2 | 127 | 138 |
| 3 | 128 | 117 |
| 4 | 101 | 98 |
| 5 | 182 | 180 |

These results show that pH is critical to achieving selectivity for LDL Cholesterol. Emulgen B66 gives selectivity for non-LDL at pH 7 but does not give selectivity at pH 5. Agreement between Reference and Measured LDL decreased if other reaction time intervals were used. It was found that at 75 seconds, substantially all of the non-LDL Cholesterol had reacted but little or no LDL Cholesterol had reacted. At longer measurement intervals, the measured values of LDL Cholesterol decreased due to the slow but significant reaction of LDL Cholesterol in the presence of Emulgen B66. Thus, in the spectrophotometric assay, control of reaction time is critical to the measurement of LDL Cholesterol

### Example 2

### Spectrophotometric Assay of Elevated LDL Samples in Liquid

We extended the observations in Example 1 to additional samples with elevated LDL Cholesterol. Total Cholesterol was run as above with Reagent 1a (pH 5/Triton X100) and Reagent 2. Non-LDL was measured with Reagent 1c (pH 7/TritonX100) and Reagent 2. Readings were taken 75 seconds after initiating the reactions with Reagent 2.

| Sample | Reference LDL | Measured LDL |
|---|---|---|
| 1 | 117 | 111 |
| 2 | 174 | 187 |
| 3 | 162 | 161 |
| 4 | 182 | 168 |

Excellent agreement was obtained between the Measured and Reference Total Cholesterol.

### Example 3

### LDL Assay with Test Strips: Single Strips for Total and Non-LDL Cholesterol

Formulations for impregnation of reaction membranes were made according to the Tables below. A Foundation Solution containing a portion of the ingredients was made and used in both the Total and Non-LDL formulations.

Test strips were assembled using test strip holders as described in co-pending provisional patent application serial No. 60/342,790. The layers in the strip holders from top to bottom are shown in FIGS. 8 and 9 and are spreading mesh 200 (Tetko); blood separation layer 202; Cytosep 1660 (untreated blank) 204 and Biodyne A membrane impregnated with the either Total Cholesterol (206) or non-LDL Cholesterol formulation (208).

Strips were assembled and tested using whole blood (EDTA anticoagulated) with a Bioscanner 2000 reader, available from Polymer Technology Systems, Inc. Indianapolis, Ind. Cholesterol concentrations were calculated using a curve set prepared using total cholesterol strips. This curve was applied to readings from both total 206 and non-LDL 208 layers. Photometric readings were made at ten second intervals over the course of the reaction.

Total cholesterol measurements were made in duplicate and averaged. Non-LDL Cholesterol measurements were performed with ten replicates and averaged. LDL Cholesterol was calculated as the difference between the Total and the Non-LDL Cholesterol.

Total Cholesterol Formulation

| | | |
|---|---|---|
| Deionized Water | 200 | g |
| Triton X-100 | 0.771 | g |
| Cholesterol Foundation** | 532 | g |
| BSA | 13.88 | g |
| 10% Gantrez AN-139 (w/v) | 95.61 | g |
| CHAPS (3-{(3-Cholamidopropyl)dimethylammonio}- | 19.82 | g |
| 1-propane-sulfonate) | | |
| Sucrose | 37.01 | g |
| pH 4.9-5.1 | | |
| Potassium Ferrocynanide | 0.116 | g |
| TOOS | 0.37 | g |
| MAOS | 4.63 | g |
| Cholesterol Oxidase | 148 | KU |
| Peroxidase | 462.6 | KU |
| Cholesterol Esterase | 92.5 | KU |
| 4-Amino antipyrine | 4.163 | g |
| Final pH 5.3-5.5 | | |
| Q.S. to 1000 mL with D. I. Water | | |

** Cholesterol Foundation:

| | | |
|---|---|---|
| D. I. Water | 800 | g |
| Sodium Citrate, dihydrate | 30 | g |
| PVP K-30 | 60 | g |
| Benzoic Acid | 2 | g |
| BSA | 4 | g |
| EDTA, disodium, dihydrate | 1.47 | g |
| pH 5.4-5.6 | | |
| Q.S. to 1000 mL with D. I. Water | | |
| Catalase | 0.05 | KU |

Non LDL Cholesterol Formulation:

| Cholesterol Foundation** | 0.532 | g/mL |
|---|---|---|
| Emulgen B66 | 8% | |
| BSA | 1.388% | |
| Gantrez AN-139 (w/v) | 0.956% | |
| Sucrose | 3.7% | |
| MOPS | 25 | mM |
| pH 7.4-7.6 | | |
| Potassium Ferrocyanide | 0.275 | mM |
| MAOS | 0.00463 | g/mL |
| Cholesterol Oxidase | 0.074 | KU/mL |
| Peroxidase | 0.2313 | U/mL |
| Cholesterol Esterase | 0.24 | KU/mL |
| 4-Amino antipyrine | 0.00416 | g/mL |
| Final pH 7.4-7.6 | | |

### Assay of LDL Cholesterol with Separate Total and Non-LDL Cholesterol Test

| Sample | Reference LDL | Measured LDL |
|---|---|---|
| 1 | 124 | 115 |
| 2 | 94 | 102 |
| 3 | 107 | 115 |
| 4 | 83 | 109 |
| 5 | 127 | 114 |
| 6 | 89 | 87 |
| 7 | 140 | 130 |
| 8 | 78 | 83 |
| 9 | 111 | 95 |
| 10 | 112 | 92 |
| 11 | 49 | 72 |
| 12 | 91 | 88 |
| 13 | 130 | 142 |
| 14 | 136 | 113 |
| 15 | 154 | 127 |
| 16 | 102 | 87 |
| 17 | 188 | 172 |

From the above date, it can be appreciated that there was excellent agreement between the reference and Measured LDL Cholesterol values. To determine the best time for endpoint of the first stage reaction, LDL cholesterol levels were taken at several different times over the course of the reaction. Remarkably, it was determined that the best results were consistently obtained when both total and non-LDL Cholestrol reactions were allowed to reach a pseudo endpoint as determined by the Bioscanner software and as explained above. As noted above, this is very surprising since the data with the spectrophotometric assay required strict control of read time and temperature.

### Example 4

### LDL Assay with Test Strips: Two Hole Test Strips for Total and Non-LDL Cholesterol

We used the same formulation for non-LDL Cholesterol as in Example 3. A modified Total Cholesterol Formulation was used:

| Cholesterol Foundation** | 0.532 | g/mL |
|---|---|---|
| CHAPS | 1.982% | |
| Emulgen B66 | 8% | |
| BSA | 1.388% | |
| Gantrez AN-139 (w/v) | 0.956% | |
| Sucrose | 3.7% | |
| MOPS | 25 | mM |
| pH 7.4-7.6 | | |
| Potassium Ferrocynanide | 0.275 | mM |
| MAOS | 0.00463 | g/mL |
| Cholesterol Oxidase | 0.074 | kU/mL |
| Peroxidase | 0.2313 | kU/mL |
| Cholesterol Esterase | 0.24 | kU/mL |
| 4-Amino anti-pyrine | 0.00416 | g/mL |
| Final pH 7.4-7.6 | | |

Strip holders with two reaction zones were used to assemble test strips with both Total and Non-LDL Cholesterol reaction pads. A cross section of the layers is shown in FIG. 10. The layers from top to bottom were a spreading layer 300 consisting of Accuflow PS (Schleicher&Schuell), blood separation layer 302 and reaction membranes 304 and 306 composed of Biodyne A impregnated with either Total Cholesterol Reagent (304) or Non-LDL Cholesterol Reagent (306). The spreading layer 300 spread the sample evenly and delivered it vertically to the blood separation layer 302, which in turn retained red blood cells before delivering plasma to reaction layers 304 and 306.

Whole blood samples (EDTA anticoagulated) were tested using the Bioscanner 2000. Separate standard curves were set for total and non-LDL cholesterol. Reference non-LDL cholesterol was determined by measuring both total and LDL cholesterol with automated methods and then subtracting the two measured values. LDL cholesterol was calculated with the test strips by subtracting the measured non-LDL from the measured total cholesterol.

### Assay of LDL Cholesterol with Two Hole Total and Non-LDL Cholesterol Test Strips

| Sample | Reference LDL | Measured LDL |
|---|---|---|
| 1 | 118 | 123 |
| 2 | 118 | 118 |
| 3 | 117 | 117 |
| 4 | 156 | 145 |
| 5 | 156 | 160 |

As shown above, there was excellent agreement between the LDL cholesterol values obtained with the two hole test strips and the Reference LDL values. This approach greatly simplifies the assay and improves precision relative to the separate test strips.

### Example 5

### LDL Assay with Test Strips: Three Hole Test Strips for Total and Non-LDL Cholesterol

Strip holders with three reaction zones were used to assemble test strips with both total and non-LDL Cholesterol reaction pads or stacks as shown in FIG. 5. The layers from top to bottom were a spreading layer 38 made from Accuflow PS (Schleicher & Schuell), blood separation layer 40 (as described above with reference to FIG. 5) and reaction membranes 92, 94 and 96 made from Biodyne A impregnated with either Total Cholesterol Reagent (92 and 96) or Non-LDL Cholesterol Reagent (94). The spreading and blood separation layers 38 and 40 covered all three stacks 92, 94 and 96. Layer 38 spreads the sample evenly and delivered it vertically downward to the blood separation layer 40, which separated red blood cells. The functions of layers 38 and 40 are described in more detail in co-pending application serial No. 60/344,300, incorporated herein by reference.

Whole blood samples (EDTA anticoagulated) were tested using the Cardiochek PA. Separate standard curves were set for total and non-LDL Cholesterol. Reference non-LDL Cholesterol was determined by measuring both total and LDL Cholesterol with automated methods and then subtracting the two measured values. LDL Cholesterol was calculated with the test strips by subtracting the measured Non-LDL from the measured Total Cholesterol. Non-LDL cholesterol reaction membrane 94 was positioned as the center panel or stack and the two outer panels 92 and 96 were impregnated with the total cholesterol solution. Results were computed with by averaging the two total cholesterol obtained from photometrically reading the color production from layers 92 and 96 or by using a single total cholesterol value from layer 92 or 96. In either case, only a single non-LDL value was obtained from the color production in layer 94.

### Assay of LDL Cholesterol with Three Hole Total and Non-LDL Cholesterol Test Strips

| | | Measured LDL (Duplicate |
|---|---|---|
| Sample | Reference LDL | TC) |
| 1 | 118 | 126 |
| 2 | 118 | 112 |
| 3 | 117 | 116 |
| 4 | 156 | 146 |
| 5 | 156 | 163 |
| Sample | Reference LDL | Measured LDL (one TC) |
| 1 | 118 | 123 |
| 2 | 118 | 117 |
| 3 | 117 | 115 |
| 4 | 156 | 144 |
| 5 | 156 | 165 |

As shown above, there was excellent agreement between the LDL cholesterol values obtained with the three hole test strips and the reference LDL values. This format holds great promise for improved lipid panel results. Substitution of one of the Total Cholesterol Reaction stacks with an HDL cholesterol stack would provide estimates of total, LDL and HDL Cholesterol in a single test. An HDL stack suitable for use with the present invention is taught and disclosed in co-pending and commonly owned provisional application serial No. 60/344,300.

### Alternate Embodiments

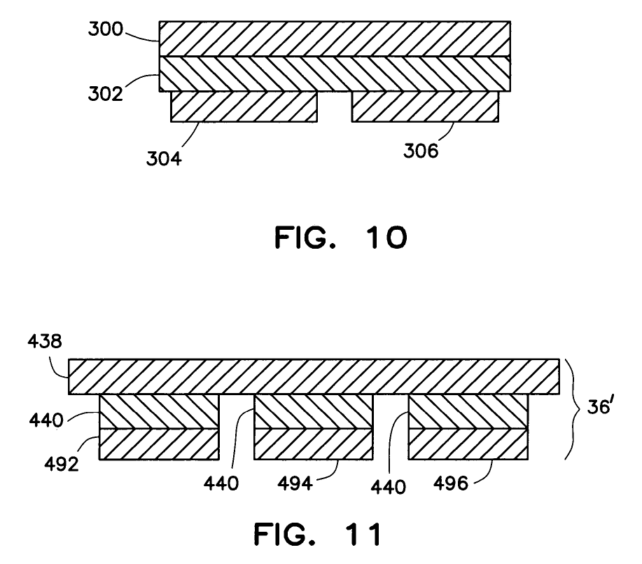

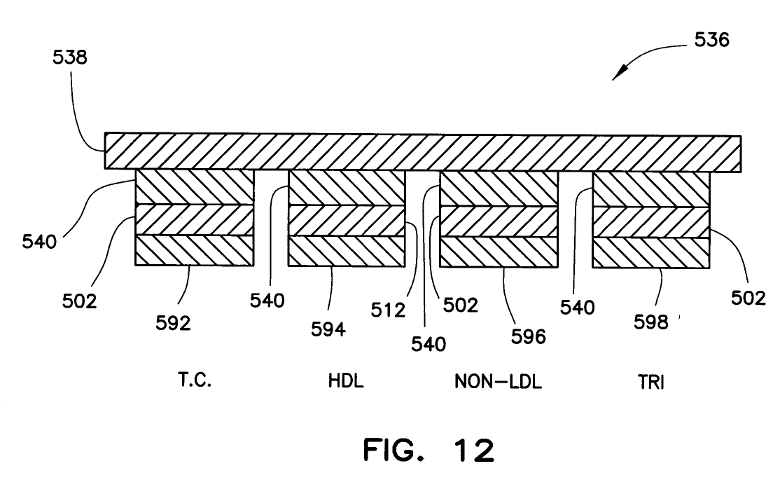

Matrix 36′ shown in FIG. 11 includes three stacks that are spaced apart and are adjacent to and in fluid communication with disbursement layer 38. Each stack has its own blood separation layer 440 as its top layer. The difference between matrix 36′ and matrix 36 (FIG. 5) is that matrix 36′ has separate blood separation layers 440 for each stack. Otherwise, matrix 36′ is the same as matrix 36 described with reference to FIG. 5. The embodiment shown in FIG. 11 is advantageous in that layers 440 collectively consume less blood than all of layer 40 (FIG. 5), which may help reduce the quantity of blood required to complete the test. Experimental data show that either the separate blood separation layers as shown in FIG. 11 or a single blood cell separation layer as shown in FIG. 5 produce accurate results.

FIG. 12 illustrates a panel including 4 stacks. One embodiment for this panel could include a total cholesterol stack, an HDL stack, a non-LDL stack and a triglycerides stack. The top layer is a spreader layer 536, as described above with reference to FIG. 5, except slightly longer so as to accommodate four stack. The first (total cholesterol), third (non-LDL) and fourth (triglycerides) stacks include a spacer layer 502, as taught in co-pending provisional application serial No. 60/344,300. The spacer layers can be formed from CytoSep 1660 and serve to keep the stacks even, since layer 512 is needed in the HDL stack to precipitate and separate non-HDLs, as also taught in co-pending application serial No. 60/344,300. The HDL and triglycerides stacks are fully disclosed and taught in co-pending application serial No. 60/344,300, as is the total cholesterol stack. The non-LDL stack is only disclosed in this application.

Advantageously, the four-stack system just described can incorporate an error checking system by incorporating the Friedewald equation described above. For example, an opto-electronic instrument such as Cardio-Chek, available from Polymer Technology Systems, Inc., can be programmed to calculate the values of total cholesterol, LDL, HDL and triglycerides in a sample being tested by strip 536. Then, using the Friedewald equation, the instrument can calculate the value of LDL and compare it to the measured value, the measured value being the measure total cholesterol value less the measured non-LDL. If the two values differ by a predetermined percentage, an error signal can be produced. For example, the display on the instrument can instruct the user to repeat the test.

Other calculations are also possible. For example, the value of VLDL cholesterol plus chylomicrons can be determined by taking the measured non-LDL value and subtracting therefrom the measured value of HDL.

As noted in co-pending provisional application 60/344,300, additional stacks for ketones, creatinine and other analytes could also be added to the test strip.

While a preferred embodiment incorporating the principles of the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A method of determining concentration of non-LDL cholesterol in a whole blood sample using a dry phase test strip, comprising:
(a) contacting the whole blood sample with a blood separation layer of the test strip and separating the blood cells from the sample, thereby producing plasma;
(b) contacting the plasma passing through the blood separation layer with a test layer and reacting the non-LDL fraction substantially faster than the LDL fraction to produce a color in the test layer substantially in proportion to the concentration of non-LDL cholesterol in the sample; and
(c) measuring the color produced in step (b).

2. The method of claim 1, wherein steps (a)-(c) are conducted at room temperature.

3. The method of claim 1, wherein step (c) is initiated by an end-point algorithm.

4. A test strip (20) for determining the concentration of LDL cholesterol in a sample of whole blood or plasma, comprising:
(a) a test strip matrix (36) having at least two stacks (92, 94);
(b) a first of said stacks (92) having reagents incorporated therein to produce a calorimetric response in proportion to the amount of total cholesterol in the sample; and
(c) a second of said stacks (94) having reagents incorporated therein to produce a colorimetric response in proportion to the amount of non-LDL cholesterol in the sample,
whereby, the value of non-LDL cholesterol obtained from said second of said stacks can be subtracted from the value of total cholesterol obtained from said first of said stacks to yield the value of LDL cholesterol in the sample.

5. A test strip (20) for determining the concentration of non-LDL cholesterol in a sample of whole blood or plasma, comprising:
a test strip matrix (36) having at least two layers (40, 42) facing and in fluid communication with one another;
a first of said layers (40) separating blood cells from plasma; and
a second of said layers (42) having reagents incorporated therein to produce a colorimetric response in proportion to the amount of non-LDL cholesterol in a sample of plasma delivered thereto.

6. The test strip of claim 5, wherein said second layer includes a surfactant that acts on non-LDLs.

7. The test strip of claim 6, wherein said surfactant includes emulgen B66.
